(19) United States
(12) Patent Application Publication
(10) Pub. No.: US 2024/0062471 A1
(43) Pub. Date: Feb. 22, 2024

(54) IMAGE PROCESSING APPARATUS, ENDOSCOPE APPARATUS, AND IMAGE PROCESSING METHOD

(71) Applicant: OLYMPUS MEDICAL SYSTEMS CORP., Tokyo (JP)

(72) Inventors: Hiroshi TANAKA, Tokyo (JP); Yamato KANDA, Tokyo (JP); Makoto KITAMURA, Tokyo (JP); Takehito HAYAMI, Tokyo (JP)

(73) Assignee: OLYMPUS MEDICAL SYSTEMS CORP., Tokyo (JP)

(21) Appl. No.: 18/385,532

(22) Filed: Oct. 31, 2023

Related U.S. Application Data
(63) Continuation of application No. PCT/JP2021/026430, filed on Jul. 14, 2021.

(51) Int. Cl. G06T 19/00; A61B 1/00; A61B 34/10; G06T 7/73

(52) U.S. Cl. CPC G06T 19/00; A61B 1/00045; A61B 34/10; G06T 7/75; A61B 2034/105; G06T 2207/10068; G06T 2207/30004; G06T 2210/41

(57) ABSTRACT

An image processing apparatus includes a processor. The processor is configured to acquire image information from an endoscope during observation of an inside of a subject, generate an organ model from the acquired image information, identify an unobserved region that is not observed by the endoscope in the organ model, estimate a top and a bottom and an azimuth of an image pickup visual field of the endoscope with respect to the organ model, set a display direction of the organ model based on the top and the bottom and the azimuth of the image pickup visual field, and output the organ model to the monitor, the organ model being associated with the identified unobserved region.

### CROSS REFERENCE TO RELATED APPLICATION

This application is a continuation application of PCT/JP2021/026430 filed on Jul. 14, 2021, the entire contents of which are incorporated herein by this reference.

### BACKGROUND OF THE INVENTION

### 1. Field of the Invention

The present invention relates to an image processing apparatus, an endoscope apparatus, and an image processing method for controlling display of an unobserved region.

### 2. Description of the Related Art

In recent years, an endoscope system using an endoscope has been widely used in a medical field and in an industrial field. For example, in the medical field, an endoscope may be inserted into an organ having a complex luminal shape inside a subject, and be used for detailed observation and examination of the inside of the organ. Some of such endoscope systems have a function that allows an operator to grasp which site in a luminal organ the operator has observed with the endoscope.

For example, in order to present a region observed with an endoscope, some endoscope systems obtain a shape of inner cavities of an organ from an endoscopic image obtained by image pickup with the endoscope, generate a three-dimensional shape model image on the spot, and display an observation position on the generated three-dimensional shape model image during observation.

Japanese Patent Application Laid-Open Publication No. 2020-154234 discloses a technology for displaying, during observation such as prescribed examination with an endoscope, a region having been observed (hereinafter referred to as an observed region) and a region not having been observed (hereinafter referred to as an unobserved region) on a three-dimensional shape model image in an identifiable manner. According to the proposal in Japanese Patent Application Laid-Open Publication No. 2020-154234, the unobserved region is displayed on a three-dimensional model or within an examination screen of a monitor that displays an examination image acquired with the endoscope. The display inside the examination screen and the display on the three-dimensional shape model image allows an operator to grasp to some extent, for example, which position in the body into which an endoscope is inserted is under observation and to confirm whether or not observation of all the regions in a subject body is completed.

### SUMMARY OF THE INVENTION

An image processing apparatus according to one aspect of the present invention includes a processor. The processor is configured to acquire image information from an endoscope during observation of an inside of a subject, generate an organ model from the acquired image information, identify an unobserved region that is not observed by the endoscope in the organ model, estimate a top and a bottom and an azimuth of an image pickup visual field of the endoscope with respect to the organ model, set a display direction of the organ model based on the top and the bottom and the azimuth of the image pickup visual field, and output the organ model to a monitor, the organ model being associated with the unobserved region identified.

An endoscope apparatus according to one aspect of the present invention includes an endoscope, an image processing apparatus including a processor, and a monitor. The processor is configured to acquire image information from the endoscope during observation of an inside of a subject, generate an organ model from the acquired image information, identify an unobserved region that is not observed by the endoscope in the organ model, estimate a position and posture of the endoscope with respect to the organ model, set a display direction of the organ model based on the position and posture of the endoscope, and output the organ model to the monitor, the organ model being associated with the unobserved region identified.

An image processing method according to one aspect of the present invention includes acquiring image information from an endoscope during observation of an inside of a subject, generating an organ model from the image information acquired, identifying an unobserved region that is not observed by the endoscope in the organ model, estimating a position and posture of the endoscope with respect to the organ model, setting a display direction of the organ model based on the position and posture of the endoscope, and outputting the organ model to a monitor, the organ model being associated with the unobserved region identified.

### Advantageous Effect of Invention

The present invention has an effect that the position of an unobserved region can be easily grasped.

### BRIEF DESCRIPTION OF THE DRAWINGS

FIG. 1 is a schematic configuration diagram showing an endoscope apparatus including an image processing apparatus according to a first embodiment of the present invention;

FIG. 2 is a perspective view showing a configuration of an endoscope in FIG. 1;

FIG. 3 is a block diagram showing an example of a specific configuration of a processor 20 in FIG. 1:

FIG. 4 is an explanatory view for describing position and posture estimation processing and organ model generation processing by a position and posture estimation unit 24 and a model generation unit 25;

FIG. 5 is a flowchart showing Visual SLAM (simultaneous localization and mapping) processing using a publicly-known Structure from Motion (SfM) shown in FIG. 4;

FIG. 6 is an explanatory view for describing organ model display;

FIG. 7 is an explanatory view for describing the organ model display;

FIG. 8 is an explanatory view for describing how to obtain a position and posture of a distal end portion 33c:

FIG. 9 is a flowchart for describing operation in the first embodiment;

FIG. 10 is an explanatory view showing an example of the organ model display in the first embodiment;

FIG. 11 is an explanatory view for describing viewpoint direction control by a display content control unit 27.

FIG. 12 is a flowchart showing a modification;

FIG. 13 is an explanatory view for describing the modification in FIG. 12;

FIG. 14 is a flowchart showing a modification;

FIG. 15 is an explanatory view for describing the modification in FIG. 14;

FIG. 16 is an explanatory view showing a modification;

FIG. 17 is an explanatory view showing a modification:

FIG. 18 is a flowchart showing a modification;

FIG. 19 is a flowchart showing a second embodiment of the present invention;

FIG. 20 is an explanatory view for describing a detection method of an occlusion region:

FIG. 21 is an explanatory view for describing the detection method of the occlusion region:

FIG. 22 is an explanatory view showing an example of a display method of the occlusion region by the display content control unit 27;

FIG. 23 is an explanatory view showing an example of a display method of a photographed region by the display content control unit 27:

FIG. 24 is an explanatory view for describing an examination screen outside region:

FIG. 25 is an explanatory view showing an example of a display method of the examination screen outside region;

FIG. 26 is an explanatory view showing an example of the display method of the examination screen outside region;

FIG. 27 is an explanatory view showing a display example of various types of information about the examination screen outside region;

FIG. 28 is a flowchart showing a third embodiment of the present invention;

FIG. 29 is an explanatory view showing a state where an image of an inside of a lumen PA3 is picked up by an image pickup device 31 in the distal end portion 33c;

FIG. 30 is an explanatory view for describing viewpoint control in accordance with a distance d;

FIG. 31 is an explanatory view for describing magnification rate control in accordance with the distance d;

FIG. 32 is an explanatory view for describing emphasis display in accordance with a distance; and

FIG. 33 is an explanatory view for describing display control in accordance with an observation route.

### DETAILED DESCRIPTION OF THE PREFERRED EMBODIMENTS

Hereinafter, embodiments of the present invention will be described in details with reference to the drawings.

### First Embodiment

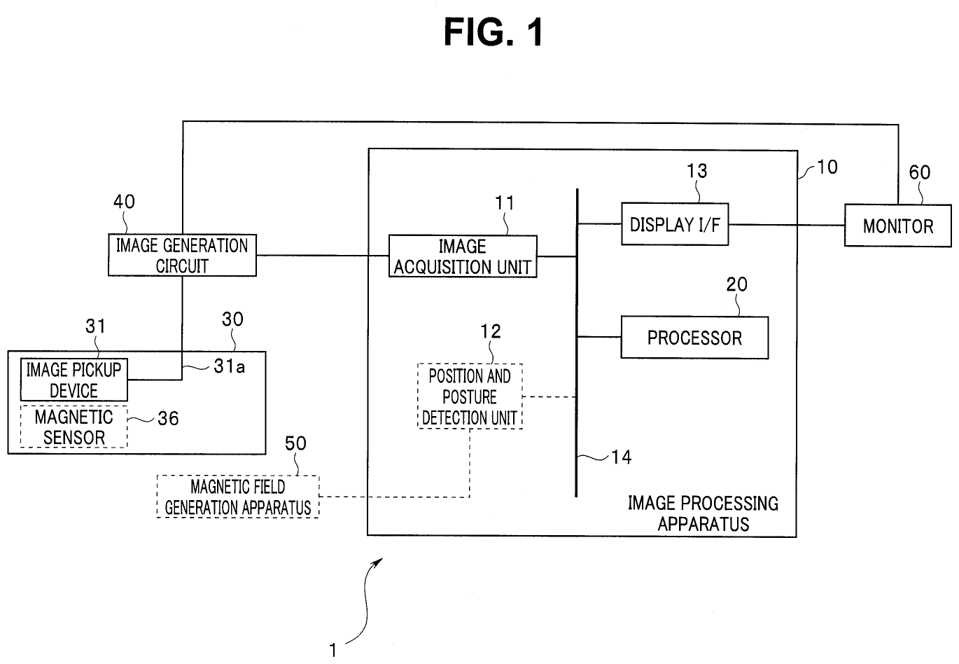

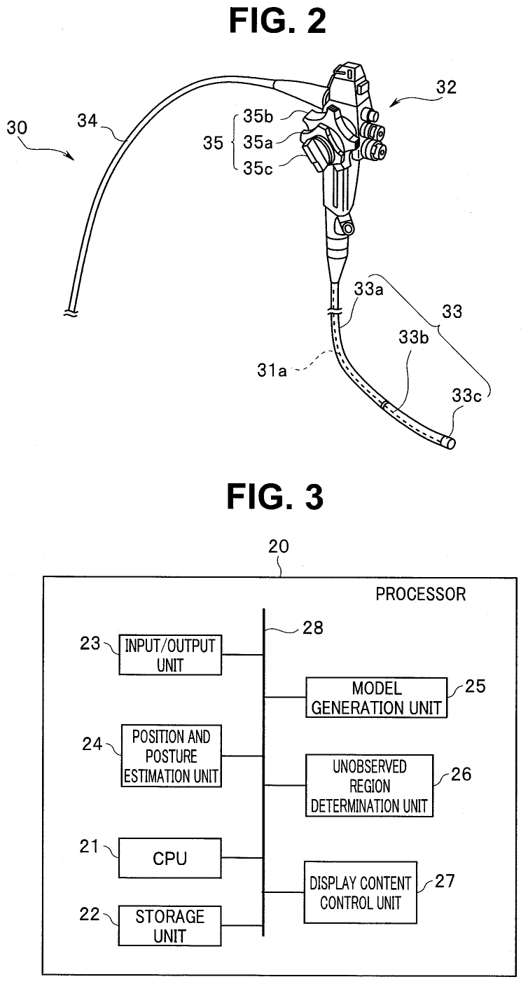

FIG. 1 is a schematic configuration diagram showing an endoscope apparatus including an image processing apparatus according to a first embodiment of the present invention. FIG. 2 is a perspective view showing a configuration of an endoscope in FIG. 1. The present embodiment is configured to determine an unobserved region and display the unobserved region on an organ model in an easy-to-understand manner based on a top and bottom of an examination screen by the endoscope. In the present embodiment, it is also possible to observe the inside of a subject with the endoscope and generate a three-dimensional shape organ model by using an examination image obtained by the endoscope at the time of the observation. In this case, as the observation progresses, the organ model is sequentially constructed and displayed up to an observation range. In other words, in this case, constructed regions of the organ model are observed regions, and not-constructed regions are unobserved regions. Among the unobserved regions, a region surrounded with the observed regions is defined as an unobserved region in a narrow sense, and the unobserved region in the narrow sense is treated as the unobserved region in the specification described below.

In the present embodiment, the unobserved region may be displayed in an easy-to-understand manner on the three-dimensional shape organ model that is already generated before observation. Note that existing organ models may be organ models generated in previous examinations or observations, or general-purpose organ models created for a prescribed luminal organ or the like. The present embodiment is applicable to both the cases where the organ model is already created before observation and where the organ model is created concurrently with the observation.

As shown in FIG. 1, the endoscope apparatus 1 includes an image processing apparatus 10, an endoscope 30, an image generation circuit 40, a magnetic field generation apparatus 50, and a monitor 60. Note that the magnetic field generation apparatus 50 can be omitted. As shown in FIG. 2, the endoscope 30 includes an operation portion 32, an insertion portion 33 having flexibility, and a universal cable 34 including signal lines. The endoscope 30 is a tubular insertion apparatus having a tubular insertion portion 33 that is inserted into a body cavity, the insertion portion 33 being inserted into, for example, a large intestine to pick up an image of the inside of the body cavity. At the distal end of the universal cable 34, a connector is provided, and the connector removably connects the endoscope 30 to the image generation circuit 40. Note that a light guide, which is not shown, is inserted into the universal cable 34 and the insertion portion 33, and the endoscope 30 is configured to output illumination light from a light source device, which is not shown, from the distal end of the insertion portion 33 through the light guide.

The insertion portion 33 includes a flexible tube portion 33a, a bending portion 33b that is bendable, and a distal end portion 33c from the proximal end to the distal end of the insertion portion 33. The insertion portion 33 is inserted into a lumen of a patient to be a subject. A proximal end portion of the distal end portion 33c is connected to a distal end of the bending portion 33b, and a proximal end portion of the bending portion 33b is connected to a distal end of the flexible tube portion 33a. The distal end portion 33c is a distal end portion of the insertion portion 33, that is, a distal end portion of the endoscope 30, which is a distal end rigid portion that is rigid.

The bending portion 33b is bendable in a desired direction in response to an operation performed on a bending operation member 35 (a left and right bending operation knob 35a and an upper and lower bending operation knob 35b) provided in the operation portion 32. The bending operation member 35 additionally includes a fixing knob 14c that fixes a position of the bent bending portion 33b. When an operator bends the bending portion 33b in various directions while inserting the insertion portion 33 into the large intestine or pulling the insertion portion 33 from the large intestine, the operator can thoroughly observe the large intestine of the patient. Note that the operation portion 32 is provided with various operation buttons, such as a release button and an air/water feeding button, in addition to the bending operation member 35.

In the present embodiment, a direction in which the distal end portion 33c of the insertion portion 33 (hereinafter also referred to as a distal end of the endoscope) moves (bends) when upward operation is performed by the upper and lower bending operation knob 35b is defined as a top (upper) direction. A direction in which the distal end of the endoscope moves (bends) when downward operation is performed by the upper and lower bending operation knob 35b is defined as a bottom (lower) direction. A direction in which the distal end of the endoscope moves (bends) when rightward operation is performed by the left and right bending operation knob 35a is defined as a right direction A direction in which the distal end of the endoscope moves (bends) when leftward operation is performed by the left and right bending operation knob 35a is defined as a left direction.

At the distal end portion 33c of the insertion portion 33, an image pickup device 31 is provided as an image pickup apparatus. During image pickup, illumination light from the light source device is directed by the light guide to irradiate a subject through an illuminating window (not shown) provided on a distal end surface of the distal end portion 33c. Reflected light from the subject is incident on an image pickup surface of the image pickup device 31 through an observation window (not shown) provided on the distal end surface of the distal end portion 33c. The image pickup device 31 obtains an image pickup signal by photoelectrically converting an optical image of the subject that is incident on the image pickup surface via an image pickup optical system which is not shown. The image pickup signal is supplied to the image generation circuit 40 via a signal line, which is not shown, in the insertion portion 33 and the universal cable 34.

The image pickup device 31 is fixed to the distal end portion 33c of the insertion portion 33 in the endoscope 30, and a top and bottom moving direction at the distal end of the endoscope matches with a vertical scanning direction of the image pickup device 31. In other words, the image pickup device 31 is arranged so that a start side of vertical scanning by the image pickup device 31 is matched with a top direction (upward direction) at the distal end of the endoscope, and an end side of the vertical scanning matches with a bottom direction (downward direction) at the distal end of the endoscope. In other words, the top and the bottom of an image pickup visual field of the image pickup device 31 match with the top and the bottom of the distal end of the endoscope (the distal end portion 33c), respectively. In addition, the top and the bottom of the image pickup device 31, that is, the top and the bottom of the distal end of the endoscope, match with the top and the bottom (the upper and lower sides) of an examination image based on the image pickup signal from the image pickup device 31, respectively.

The image generation circuit 40 is a video processor that performs prescribed image processing on a received image pickup signal and generates an examination image. The image pickup signal of the generated examination image is outputted from the image generation circuit 40 to the monitor 60, so that a live examination image is displayed on the monitor 60. For example, in a case of performing examination of a large intestine, a doctor who performs the examination can insert the distal end portion 33c of the insertion portion 33 through an anus of a patient and observe the inside of the large intestine of the patient with use of the examination image displayed on the monitor 60.

The image processing apparatus 10 includes an image acquisition unit 11, a position and posture detection unit 12, a display interface (hereinafter referred to as an I/F) 13, and the processor 20. The image acquisition unit 11, the position and posture detection unit 12, the display I/F 13, and the processor 20 are connected to each other by a bus 14.

The image acquisition unit 11 takes in examination images from the image generation circuit 40. The processor 20 takes in the examination images via the bus 14. Based on the taken-in examination images, the processor 20 detects an unobserved region, generates an organ model, and generates display data for displaying an image indicating the unobserved region that is superimposed on the organ model. The display I/F 13 takes in the display data from the processor 20 via the bus 14, converts the data to a format that can be displayed on a display screen of the monitor 60, and then outputs the data to the monitor 60.

The monitor 60 as a notification unit displays the examination image from the image generation circuit 40 on the display screen, and displays the organ model from the image processing apparatus 10 on the display screen. For example, the monitor 60 may include a PinP (picture in picture) function, so that both the examination image and the organ model can be displayed at the same time. The notification unit is not limited to notification means using visual information, and may be configured to notify position information by voice or issue operation instructions, for example.

In the present embodiment, the processor 20 creates display data so that the operator can easily grasp the position of the unobserved region.

FIG. 3 is a block diagram showing an example of a specific configuration of the processor 20 in FIG. 1.

The processor 20 includes a central processing unit (hereafter referred to as a CPU) 21, a storage unit 22, an input/output unit 23, a position and posture estimation unit 24, a model generation unit 25, an unobserved region determination unit 26, and a display content control unit 27. The storage unit 22 is constituted of, for example, a ROM, a RAM or the like. The CPU 21 operates according to a program stored in the storage unit 22 to control each unit of the processor 20 and the image processing apparatus 10 as a whole.

Note that the position and posture estimation unit 24, the model generation unit 25, the unobserved region determination unit 26, and the display content control unit 27 included in the processor 20 may include a CPU not shown, and the CPU may operate according to programs stored in the storage unit 22 to implement desired processing, or some or all of the respective functions may be implemented by an electronic circuit. The CPU 21 may be configured to implement all the functions of the processor 20.

The input/output unit 23 is an interface to taken in the examination images at a fixed cycle. The input/output unit 23 acquires, for example, the examination images at a frame rate of 30 fps. Note that the frame rate of the examination images taken in by the input/output unit 23 is not limited to 30 fps.

The position and posture estimation unit 24 takes in the examination images via the bus 28 and estimates the position and the posture of the image pickup device 31. The model generation unit 25 takes in the examination images via the bus 28 and generates an organ model. Since the image pickup device 31 is fixed to the distal end side of the distal end portion 33c, it may be said that the position and posture of the image pickup device 31 is the position and the posture of the distal end portion 33c. It may also be said that the position and the posture of the image pickup device 31 is the position and the posture of the distal end of the endoscope.

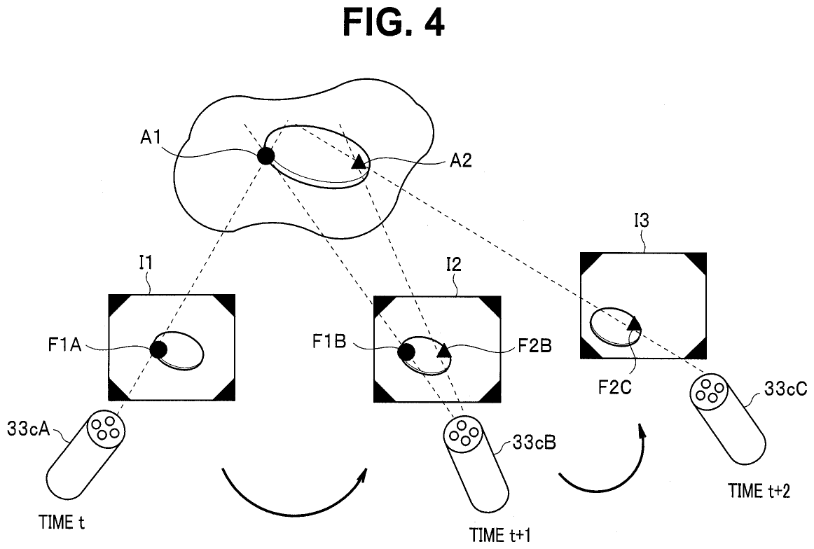

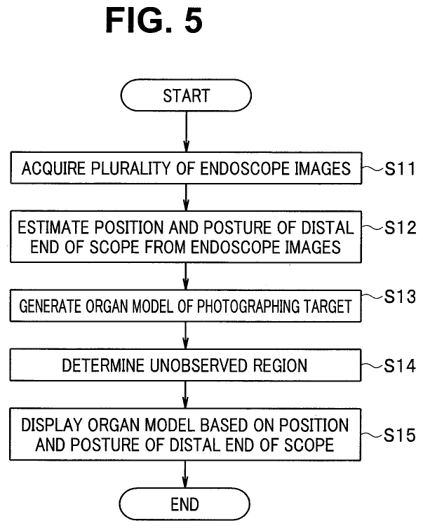

FIG. 4 is an explanatory view for describing position and posture estimation processing (hereinafter referred to as “tracking”) and organ model generation processing by the position and posture estimation unit 24 and the model generation unit 25. FIG. 5 is a flowchart showing Visual SLAM (simultaneous localization and mapping) processing using a publicly-known Structure from Motion (SfM) shown in FIG. 4.

Using Visual SLAM makes it possible to estimate the position and the posture of the image pickup device 31, that is, the position and the posture of the distal end portion 33c (the position and the posture of the distal end of the endoscope) and also makes it possible to generate the organ model. Since Visual SLAM using SfM makes it possible to acquire the position and the posture of the image pickup device 31 and a three-dimensional image of the subject, that is, an organ model, description is given on the premise, for the convenience of description, that the CPU 21 implements the functions of the position and posture estimation unit 24 and the model generation unit 25 by processing programs.

First, the CPU 21 performs initialization. It is assumed that setting values of each portion of the endoscope 30 relating to the position and posture estimation are already known by calibration to the CPU 21. By initialization, the CPU 21 also recognizes an initial position and posture of the distal end portion 33c.

In step S11 of FIG. 5, the CPU 21 sequentially takes in examination images from the endoscope 30. The CPU 21 detects feature points of the acquired examination images and attention points corresponding to the feature points. As shown in FIG. 4, the image pickup device 31 of the endoscope 30 is assumed to acquire an examination image I1 at time t. Hereinafter, the distal end portions 33c at time t, t+1, and t+2 are assumed to be distal end portions 33cA, 33cB, and 33cC, respectively. It is assumed that when image pickup by the image pickup device 31 continues while the insertion portion 33 is being moved, the image pickup device 31 acquires an examination image I2 at the position of the distal end portion 33cB at time t+1, and the image pickup device 31 acquires an examination image I3 at the position of the distal end portion 33cC at time t+2. Note that during an image pickup period of the image pickup device 31 in which the CPU 21 performs the position and posture estimation processing and the organ model generation processing, optical characteristics of the image pickup device 31, such as focal length, distortion aberration, and pixel size, are assumed to be constant.

The examination images I1, I2, . . . are sequentially supplied to the CPU 21, and the CPU 21 detects feature points from each of the examination images I1, I2 . . . For example, the CPU 21 can detect as feature points corner portions and edge portions where luminance gradients are equal to or more than a prescribed threshold in the images. The example in FIG. 4 shows that a feature point F1A is detected in the examination image I1, and a feature point F1B corresponding to the feature point F1A of the examination image I1 is detected in the examination image I2. The example in FIG. 4 shows that a feature point F2B is detected in the examination image I2, and a feature point F2C corresponding to the feature point F2B of the examination image I2 is detected in the examination image I3. Note that the number of feature points detected from each of the examination images is not particularly limited.

The CPU 21 finds corresponding feature points by collating each feature point in an examination image with each feature point in other examination images. The CPU 21 acquires the coordinates of the feature points (a pair of feature points) associated with each other in two examination images (the positions in the examination images), and calculates the position and the posture of the image pickup device 31 based on the acquired coordinates (step S12). Note that in calculation (tracking) of the position and the posture, the CPU 21 may use a basic matrix that holds relative positions and postures among the distal end portions 33cA, 33cB, . . . , that is, relative positions and postures among the image pickup devices 31 that have acquired the respective examination images.

The positions and the postures of the image pickup devices 31 are interrelated with the attention points corresponding to the feature points in the examination images, so that when one is known, the other can be estimated. The CPU 21 executes three-dimensional shape reconstruction processing of a subject based on the relative positions and postures of the image pickup devices 31. In other words, by using the corresponding feature points in the examination images obtained by the respective image pickup devices 31 at the distal end portions 33cA, 33cB, . . . , of which positions and postures are already known, the CPU 21 obtains the positions (hereinafter referred to as attention points) corresponding to the respective feature points on a three-dimensional image, based on the principle of triangulation (hereinafter referred to as mapping). The example of FIG. 4 shows that the feature points F1A and F1B are obtained as an attention point A1 in the three-dimensional image, and the feature points F2B and F2C are obtained as an attention point A2 in the three-dimensional image. Note that various methods can be adopted as a method for the CPU 21 to reconstruct a three-dimensional image. For example, the CPU 21 may adopt matching processing by PMVS (patch-based multi-view stereo) and stereo rectification, or the like.

The CPU 21 repeats tracking and mapping with use of the examination images that are picked up and obtained by the image pickup device 31 while the image pickup device 31 is moving, so as to acquire image data on the organ model that is a three-dimensional image (step S13). Thus, the position and posture estimation unit 24 sequentially estimates the position and the posture of the distal end portion 33c (the position of the distal end of the endoscope), and the model generation unit 25 sequentially creates the organ model.

The unobserved region determination unit 26 detects an unobserved region in the organ model generated by the model generation unit 25 (step S14) and outputs the information on the position of the unobserved region on the organ model to the display content control unit 27. Note that the unobserved region determination unit 26 detects a region surrounded with the organ model that is sequentially generated by the model generation unit 25 as an unobserved region. The display content control unit 27 receives image data from the model generation unit 25 and also receives the information on the position of the unobserved region from the unobserved region determination unit 26. The display content control unit 27 generates and outputs display data so as to display an organ model display that is obtained by synthesizing an image indicating the unobserved region on the image of the organ model. In this way, the organ model on which the image of the unobserved region is superimposed is displayed on the display screen of the monitor 60 (step S15).

(Relation of Position and Posture of Image Pickup Device 31 with Examination Screen and Organ Model Display)

As described above, the image pickup device 31 is fixed to the distal end portion 33c of the insertion portion 33 in the endoscope 30, and the top and the bottom of the distal end of the endoscope in the moving direction match with the top and the bottom of the image pickup device 31, respectively. The top and the bottom of the examination image acquired by the image pickup device 31 also match with the top and the bottom of the image pickup device 31 (the distal end of the endoscope) in the moving direction, respectively. The examination image acquired by the image pickup device 31 is subjected to image processing and is then supplied to the monitor 60. In the present embodiment, the terms “the top and the bottom of the distal end of the endoscope”, “the top and the bottom of the distal end portion 33c”, and “the top and the bottom of the image pickup device 31” are used synonymously. The terms “the position and the posture of the distal end of the endoscope”, “the position and the posture of the distal end portion 33c”, and “the position and the posture of the image pickup device 31” are also used synonymously.

The monitor 60 displays an examination image on the display screen. In the display screen of the monitor 60, an image displayed in a region where the examination image is displayed is defined as an examination screen. The top and bottom direction of the examination screen matches with a vertical scanning direction of the monitor 60, and the start side of vertical scanning (the top of the display screen) is defined as the top of the examination screen, and the end side of the vertical scanning (the bottom of the display screen) is defined as the bottom of the examination screen. The monitor 60 matches the top and the bottom of the examination image with the top and the bottom of the display screen for display. In other words, the top and the bottom of the examination image match with the top and the bottom of the examination screen, respectively. Therefore, the top and the bottom of the distal end portion 33c of the endoscope in the direction of movement by the upper and lower bending operation knob 35b match with the top and the bottom of the examination screen, respectively. However, the top and the bottom of the organ model display may not match with the top and the bottom of the examination screen.

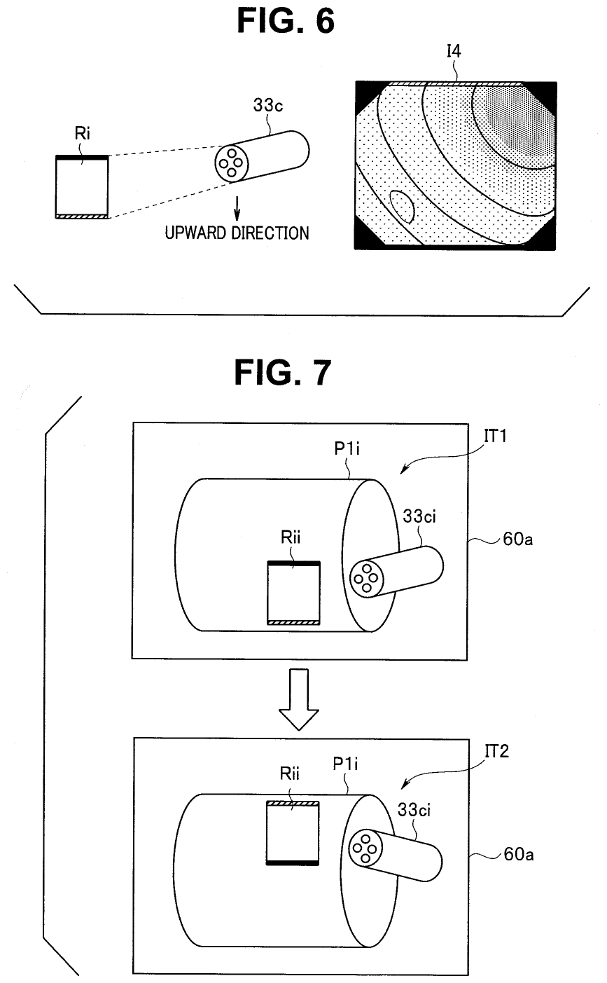

FIGS. 6 and 7 are explanatory views for describing the organ model display. FIG. 6 shows the relation between a photographing region and the direction of the distal end portion 33c of the image pickup device 31, and also shows the examination screen. FIG. 7 shows the organ model displayed on a display screen 60a of the monitor 60 and a photographing region Ri corresponding to FIG. 6. In FIGS. 6 and 7, a hatched part and a painted part in the photographing region Ri, an examination screen I4, and the display screen 60a indicate the directions corresponding to the top (upper side) or the bottom (lower side) of the distal end of the endoscope.

The example on a left side in FIG. 6 shows that a given photographing region Ri in the body is photographed by the image pickup device 31. On the left side in FIG. 6, the upward direction of the distal end of the endoscope (the distal end portion 33c) corresponds to the downward direction on the page of FIG. 6. A right side in FIG. 6 shows the examination screen I4 obtained by photographing the photographing region Ri. As described above, the top and the bottom of the examination screen I4 match with the top and the bottom of the distal end of the endoscope, respectively. Therefore, an operator can relatively easily recognize the direction in which the endoscope should be operated by referring to the examination screen I4. For example, when the operator wants to photograph a region of the subject that corresponds to a position in an upward direction on the page of FIG. 6 rather than the upper end of the examination screen I4 on the display screen of the monitor 60, the operator may simply operate the upper and lower bending operation knob 35b in the upward direction.

Organ model displays IT1 and IT2 in FIG. 7 show an organ model P1i displayed on the display screen 60a of the monitor 60. The organ model P1i is created based on a prescribed lumen of the human body. FIG. 7 shows the organ model displays IT1 and IT2 in a state where the image pickup device 31 picks up images of the photographing region Ri in the lumen corresponding to the organ model P1i, in a photographing state shown on the left in FIG. 6. In FIG. 7, the upward direction on the page corresponds to the upward direction of the display screen 60a. In other words, the organ model display IT1 is displayed in a state where the top and the bottom of an image Rii in the photographing region on the display screen 60a (the top and the bottom of the distal end of the endoscope) are opposite to the top and the bottom of the display screen 60a.

Therefore, if the operator should wish to photograph an upper region on the page rather than the photographing region Ri in the lumen corresponding to the organ model P1i in FIG. 7, the operator needs to operate the upper and lower bending operation knob 35b in a downward direction, which makes it hard for the operator to intuitively grasp the operating direction of the endoscope 30.

Therefore, in the present embodiment, the display content control unit 27 is configured to display the image of the organ model P1i by rotating the image of the organ model P1i so that the top and the bottom of the examination screen are matched with the top and the bottom of the image of the organ model P1i, respectively. Note that the top and the bottom (the upper side and the lower side) of the organ model are defined based on the top and the bottom of the examination screen currently provided by the image pickup device 31 and arranged in the organ model image. In other words, the display content control unit 27 displays an organ model image by rotating the organ model image so that the top and the bottom of the examination screen I4 in FIG. 6 match with the top and the bottom of the examination screen in the organ model on the display screen 60a, respectively. Note that the display content control unit 27 can rotate the image to be displayed around an X axis, a Y axis, and a Z axis by publicly-known image processing, for example.

The display content control unit 27 displays the organ model display IT2 in a lower stage of FIG. 7, which is obtained by rotating the organ model P1i in an upper stage of FIG. 7, on the display screen 60a. As is clear from a comparison between the top and the bottom of the examination screen I4 in FIG. 6 and the top and the bottom of the image Rii in the photographing region in the organ model P1i, the organ model display IT2 in FIG. 7 is displayed in a state where the top and the bottom of the distal end of the endoscope match with the top and the bottom of the organ model (the top and the bottom of the examination screen), respectively. Therefore, if the operator wishes to photograph a region of the lumen that corresponds to the lower side on the page rather than the image Rii in the photographing region in the organ model P1i in FIG. 7, the operator may simply operate the upper and lower bending operation knob 35b in the downward direction, which makes it easy for the operator to intuitively grasp the operation direction of the endoscope 30 from the organ model display IT2.

In this way, the display content control unit 27 creates the display data in which the organ model image is arranged so as to match the top and the bottom of the examination image with the top and the bottom of the organ model, respectively. As a result, even in the unobserved region determined by the unobserved region determination unit 26, the top and the bottom of the distal end portion 33c of the endoscope match with the top and the bottom of the display of the unobserved region on the organ model, respectively. Therefore, the organ model display allows the operator to easily and intuitively recognize the position of the unobserved region.

### (Other Examples of Position Detection)

In the above description, an example of detecting the position and the posture of the distal end portion 33c by image processing has been described. However, other methods may be employed to detect the position and the posture of the distal end portion 33c. For example, a method using a magnetic sensor may be considered. For example, a magnetic sensor 36 shown by a dashed line in FIG. 1 is arranged at the distal end portion 33c of the insertion portion 33. The magnetic sensor 36 is a detector arranged in the vicinity of the image pickup device 31 at the distal end portion 33c to detect the position and the posture of a viewpoint of the image pickup device 31. The magnetic sensor 36 includes, for example, two cylindrical coils, and two central axes of the two coils are orthogonal to each other. In other words, the magnetic sensor 36 is a six-axis sensor that detects position coordinates and a direction (i.e. an Euler angle) of the distal end portion 33c. The magnetic sensor 36 outputs a detection signal to the image processing apparatus 10.

Outside the subject in the vicinity of the magnetic sensor 36, the magnetic field generation apparatus 50 (dashed line in FIG. 1) is provided to generate a magnetic field. The magnetic field generation apparatus 50 generates a prescribed magnetic field. The magnetic sensor 36 detects the magnetic field generated by the magnetic field generation apparatus 50. The magnetic field generation apparatus 50 is connected to the position and posture detection unit 12 (dashed line) in the image processing apparatus 10 via a signal line. Thus, based on a detection result of the magnetic sensor 36, the position and posture detection unit 12 detects in real time the position and the posture of the distal end portion 33c, in other words, the position and the direction of a viewpoint of the examination image acquired by the image pickup device 31. Note that a magnetic field generating element may be provided at the distal end portion 33c in place of the magnetic sensor 36, and the magnetic sensor may be provided outside the patient in place of the magnetic field generation apparatus 50 to detect the magnetic field.

The position and posture detection unit 12 causes the magnetic field generation apparatus 50 to generate a prescribed magnetic field. The position and posture detection unit 12 detects the magnetic field by the magnetic sensor 36, and generates in real time data on position coordinates (x, y, z) and direction (i.e., an Euler angle (Ψ, θ, φ), that is, position and posture information based on a detection signal of the detected magnetic field. In other words, the position and posture detection unit 12 is a detector that detects three-dimensional arrangement including at least part of the position and direction information on the image pickup device 31, based on the detection signal from the magnetic sensor 36. More specifically, the position and posture detection unit 12 detects three-dimensional arrangement time change information that is information on changes in the three-dimensional arrangement over time. Therefore, the position and posture detection unit 12 acquires three-dimensional arrangement information on the insertion portion 33 at a plurality of time points.

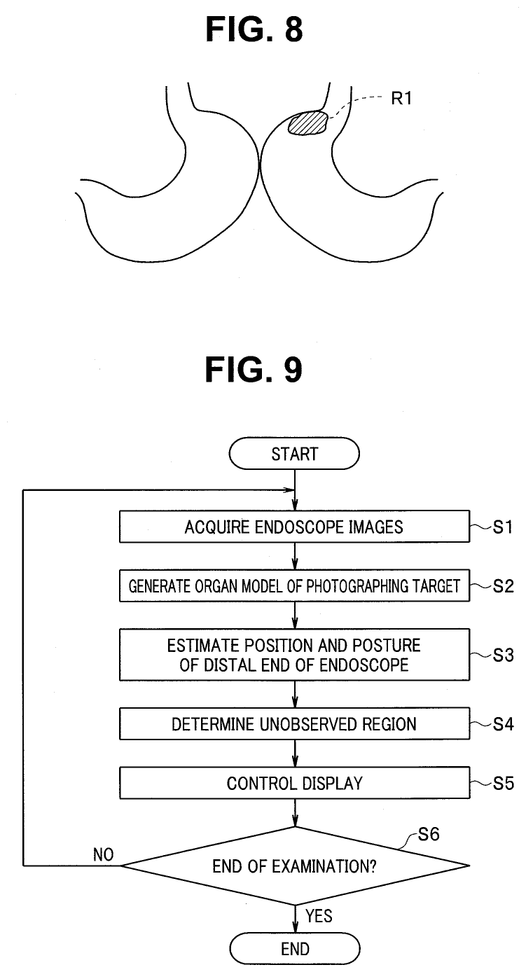

In the above description, the organ model is sequentially generated based on examination images that are sequentially inputted in the model generation unit 25. However, an existing organ model may also be used. FIG. 8 is an explanatory view for describing how to obtain the position and the posture of the distal end portion 33c in the case of using the existing organ model. The example in FIG. 8 shows a position currently observed, that is, the position of an examination image that is currently acquired by the image pickup device 31, on a schema image of the stomach. In this case, the position of the examination image currently acquired may be defined as the position of the distal end portion 33c.

Note that in the present embodiment, even in the case of using the existing organ model, the display content control unit 27 creates display data in which the organ model image is arranged so as to match the top and the bottom of the examination image with the top and the bottom of the organ model, respectively.

Next, the operation of the thus-configured embodiment is described by referring to FIGS. 9 to 10. FIG. 9 is a flowchart for describing the operation in the first embodiment, and FIG. 10 is an explanatory view showing an example of the organ model display in the first embodiment.

After power is applied to an endoscope apparatus 1, the insertion portion 33 is inserted into an examination target, and examination is started. The image pickup device 31 is driven by the image generation circuit 40 to pick up an image of the inside of the patient and acquire an endoscope image (step S1). An image signal from the image pickup device 31 is supplied to the image generation circuit 40 for prescribed image processing. The image generation circuit 40 generates an examination image (endoscope image) based on the image signal and outputs the examination image to the monitor 60. In this way, the examination image is displayed on the display screen 60a of the monitor 60.

The examination image is also supplied to the image processing apparatus 10. The image acquisition unit 11 supplies the received examination image to the processor 20. The examination image is supplied to the position and posture estimation unit 24 and the model generation unit 25 by the input/output unit 23 of the processor 20. The position and posture estimation unit 24 and the model generation unit 25 perform generation of an organ model and estimation of the position and the posture of the distal end portion 33c (the distal end of the endoscope) in steps S2 and S3. When the model generation unit 25 receives the examination images, the model generation unit 25 generates an organ model for the observed region.

The unobserved region determination unit 26 detects an unobserved region surrounded with the organ model generated by the model generation unit 25 (step S4), and outputs the determination result to the display content control unit 27.

The display content control unit 27 superimposes an image of the unobserved region on an image of the organ model from the model generation unit 25, and generates display data for matching the top and the bottom of the organ model with the top and the bottom of the distal end portion 33c, that is, the top and the bottom of the examination screen, respectively (step S5). The display data from the display content control unit 27 is supplied to the display I/F 13 via the input/output unit 23, converted to a format that can be displayed on the monitor 60, and supplied to the monitor 60. In this way, the examination screen and the organ model display including the organ model, on which the unobserved region is superimposed, are displayed on the display screen 60a of the monitor 60. The top and the bottom of the organ model and the top and the bottom of the examination screen match with the top and the bottom of the distal end of the endoscope, respectively, so that the operator can easily and intuitively grasp the position of the unobserved region from the display on the display screen 60a.

FIG. 10 shows an example of the organ model display on the display screen 66a in the above case. In an organ model display IT3 shown in FIG. 10, an image Rui of the unobserved region and an image 33ci of the distal end portion 33c are superimposed on an organ model P2i. In the organ model display IT3, the top and the bottom of the organ model P2i match with the top and the bottom of the examination screen not shown and the top and the bottom of the distal end of the endoscope, respectively. As a result, the operator can easily and intuitively grasp the position of the unobserved region from the display on the display screen 60a. For example, the operator who sees the organ model display IT3 in FIG. 10 can intuitively grasp that the unobserved region can be observed by operating the upper and lower bending operation knob 35b in the upward direction.

### (Viewpoint Direction Control)

The display content control unit 27 may further perform viewpoint direction control for the organ model.

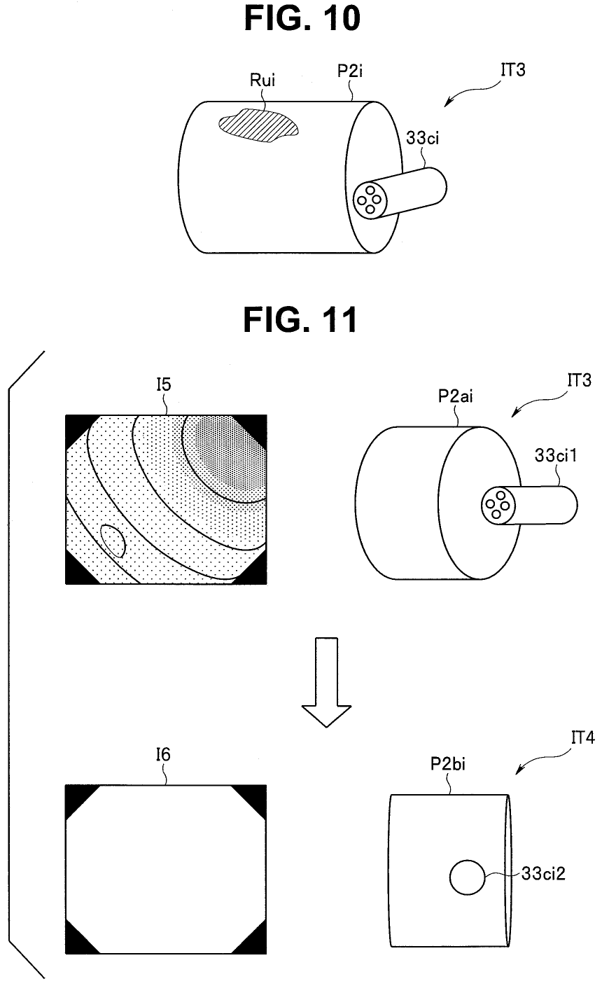

FIG. 11 is an explanatory view for describing the viewpoint direction control by the display content control unit 27. FIG. 11 shows organ model displays IT3 and IT4 when the viewpoint direction control is performed. In FIG. 11, examination screens 15 and 16 are shown on the left side, and the organ model displays IT3 and IT4 corresponding to the examination screens 15 and 16 are shown on the right side.

The examination screen I5 is obtained when the image pickup device 31 performs image pickup with an image pickup visual field in a lumen direction. In other words, a line-of-sight direction of the image pickup device 31 (an optical axis direction of an image pickup optical system) is directed in the lumen direction. In the organ model display IT3, an image 33ci1 of the distal end portion 33c superimposed on an organ model P2ai indicates that the line-of-sight direction of the image pickup device 31 is the lumen direction. In other words, as shown on an upper right side in FIG. 11, the display content control unit 27 displays the organ model display IT3, on which the image 33ci1 indicating that the distal end side of the distal end portion 33c is directed in the lumen direction is arranged, on the display screen 60a.

The examination screen I6 is obtained when the image pickup device 31 performs image pickup with an image pickup visual field in a lumen wall direction. In this case, as shown on an lower right side in FIG. 11, the display content control unit 27 displays the organ model display IT4 on the display screen 60a, the organ model display IT4 indicating that the distal end side of the distal end portion 33c faces a lumen wall by an image 33ci2 of the distal end portion 33c that is superimposed on an organ model P2bi.

For example, assume a case where the operator operates the left and right bending operation knob 35a to bend the distal end portion 33c to the right side while the organ model display IT3 is displayed on the display screen 60a, so that the examination screen I6 and the organ model display IT4 in FIG. 11 are displayed on the display screen 60a. In this case, when the operator wishes to observe a region on the left side of the page that is at the left end of the examination screen I6, the operator may simply operate the left and right bending operation knob 35a to bend the distal end portion 33c to the left.

When the viewpoint direction control shown in FIG. 11 is performed in such a manner, the operator can easily and intuitively grasp whether the visual field of the image pickup device 31 is directed in the lumen direction or in the lumen wall direction, so that the operability of the endoscope 30 can be enhanced. It is also possible to additionally display information indicating the direction of the endoscope, such as a depth direction, and a front direction, in FIG. 11. Examples of such information may include verbal information, information using symbols such as “x” and “.”, and information using an icon imitating the endoscope.

Thus, in the present embodiment, when the top and the bottom of the organ model display to be displayed is set based on the top and the bottom of the examination screen, the position of the unobserved region can be grasped easily and intuitively. This facilitates the bending operation of the endoscope for observation of the unobserved region. In addition, the organ model display is displayed according to the viewpoint direction of the image pickup device, which makes it easier to confirm the unobserved region.

### (Modification)

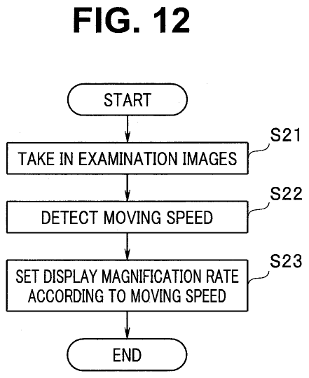

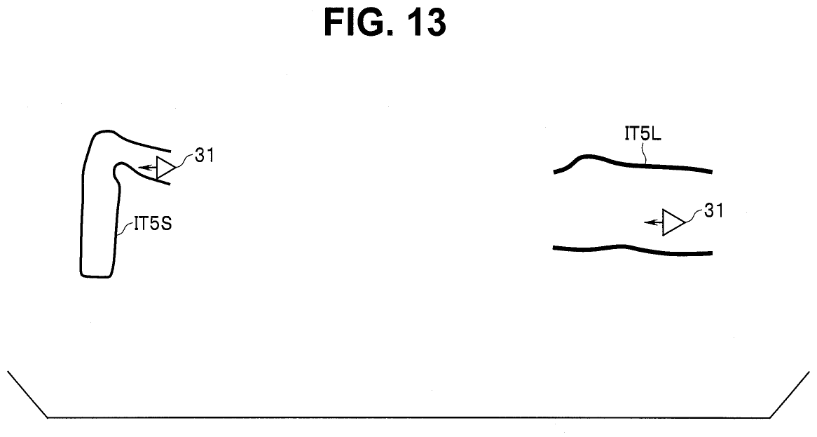

FIG. 12 is a flowchart showing a modification. FIG. 13 is an explanatory view for describing the modification in FIG. 12. A hardware configuration of the present modification is similar to the hardware configuration of the first embodiment. The modification is for changing a display magnification rate of the organ model display according to moving speed of the image pickup device 31.

The display content control unit 27 changes the display magnification rate of the organ model according to the moving speed of the image pickup device 31, in addition to display control similar to the display control in the first embodiment. In step S21 in FIG. 12, the display content control unit 27 sequentially takes in examination images, and detects the moving speed of the image pickup device 31 by image analysis of the examination images (step S22). For example, the display content control unit 27 may obtain the moving speed of the image pickup device 31 from the frame rate of the examination images and changes in position of the distal end portion 33c. The display content control unit 27 generates display data to reduce the display magnification rate of the organ model as the moving speed is faster, and to increase the display magnification rate of the organ model as the moving speed is slower (step S23). Note that the display content control unit 27 may be configured to determine the level of the moving speed of the image pickup device 31, and generate display data to reduce the display magnification rate of the organ model as the moving speed is faster and to increase the display magnification rate of the organ model as the moving speed is slower for each determined level.

FIG. 13 shows an example of changing the display magnification rate of the organ model according to the moving speed. In the example of FIG. 13, an organ model display IT5S indicates the display when the moving speed of the image pickup device 31 is a prescribed high speed, and an organ model display IT5L indicates the display when the moving speed of the image pickup device 31 is a prescribed low speed.

The organ model displays IT5S and IT5L are based on, for example, the organ model of an intestinal tract of the same subject. For example, when the operator inserts and removes the insertion portion 33 to and from the intestinal tract, the processor 20 creates the organ model of the intestinal tract. The operator examines the inside of the intestinal tract while removing the insertion portion 33 from the intestinal tract. In FIG. 13, arrows indicate an image pickup direction of the image pickup device 31. In other words, the example in FIG. 13 shows an example of displaying the organ model of a prescribed range that is mainly in the image pickup direction, in the organ model to be created.

The organ model display IT5S indicates that the organ model in a relatively wide range, from the distal end of the organ model to substantially the position of the image pickup device 31, is displayed at a relatively small display magnification rate. The organ model display IT5L indicates that the organ model in a relatively narrow range in the vicinity of the image pickup device 31 is displayed at a relatively large display magnification rate.

For example, when the insertion portion 33 is inserted and removed at relatively high speed, a relatively wide range of the organ model is displayed as in the case of the organ model display IT5S, for example, so that the movement is easily confirmed. On the contrary, in a case where, for example, a desired observation target region is confirmed in detail with the image pickup device 31, the speed of insertion and removal of the insertion portion 33 is relatively low speed, and a relatively small range of the organ model is displayed at a large magnification rate as in the case of the organ model display IT5L. This makes it possible to confirm the desired observation target region in detail.

Thus, in the modification, the organ model is displayed at a display magnification rate in accordance with the moving speed of the image pickup device 31, which makes it easy to observe the observation target region.

### (Modification)

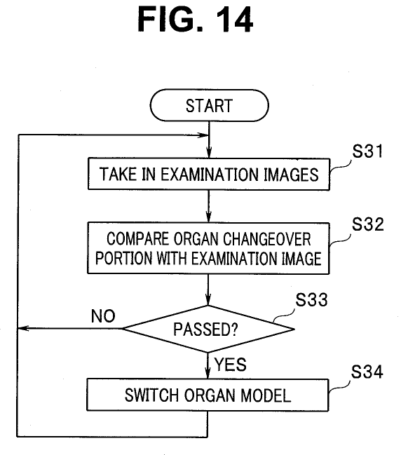

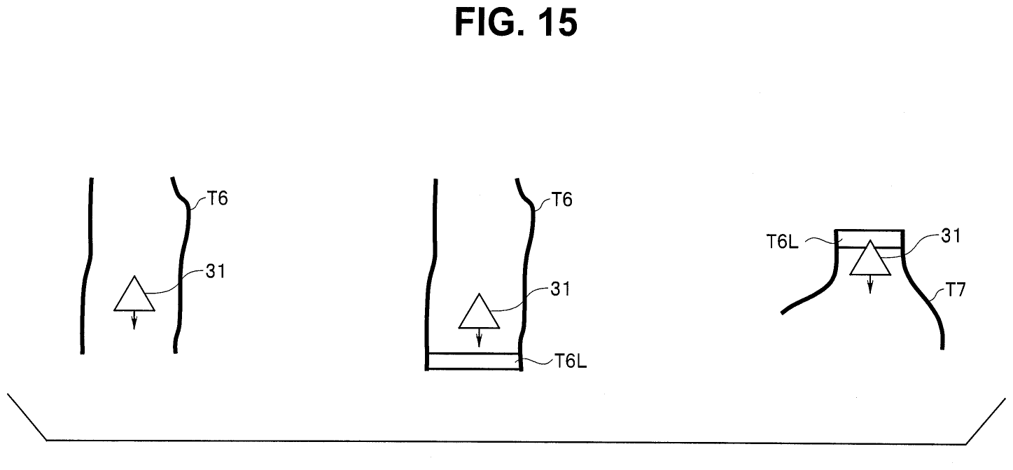

FIG. 14 is a flowchart showing a modification. FIG. 15 is an explanatory view for describing the modification in FIG. 14. A hardware configuration of the present modification is similar to the hardware configuration of the first embodiment. The present modification is to switch the organ model to be displayed when the image pickup device 31 moves between organs. Note that the direction of arrows in FIG. 15 indicates the image pickup direction of the image pickup device 31.

In addition to display control similar to the display control in the first embodiment, the display content control unit 27 also determines changeover of organs according to the examination image from the image pickup device 31, and switches the organ model display. In step S31 in FIG. 14, the display content control unit 27 takes in examination image. The display content control unit 27 compares the examination image with a changeover portion between organs (step S32) to determine whether or not the examination image is an image of the changeover portion. For example, the display content control unit 27 may use AI (artificial intelligence) to determine the changeover portion between organs. For example, an inference model is generated in advance by acquiring a plurality of examination images of parts where organs are in contact with each other (changeover portions) and performing deep learning using the examination images as teacher data. The display content control unit 27 may use the inference model to determine whether or not an examination image is about the changeover portion and obtain the result of determination.

When the display content control unit 27 detects that the distal end portion 33c (the image pickup device 31) has passed the changeover portion (step S33), the display content control unit 27 generates display data for displaying an organ model about an organ after the changeover in place of the organ model displayed before the changeover (step S34).

FIG. 15 is for describing switching of organ models. The example in FIG. 15 shows an example of a change from the organ model of an esophagus to the organ model of a stomach. An organ model T6 represents the organ model of an esophagus, and an organ model T7 represents the organ model of a stomach. For example, when the insertion portion 33 is inserted toward the stomach from the esophagus, the image pickup device 31 picks up images of the esophagus while moving in a direction indicated by an arrow in FIG. 15. As a result, as shown on a left side in FIG. 15, the organ model T6 is sequentially created based on the examination images from the image pickup device 31. When the image pickup device 31 reaches the vicinity of a changeover portion T6L, which is a boundary between the esophagus and the stomach, the image pickup device 31 picks up images of the changeover portion T6L (a center in FIG. 15). When the image pickup device 31 further moves forward in the arrow direction, the distal end portion 33c passes the changeover portion T6L. Consequently, as shown on a right side in FIG. 17, the display content control unit 27 detects that the distal end portion 33c has passed the changeover portion T6L between the esophagus and the stomach, switches the model image to be displayed to the organ model T7, and displays the organ model T7.

Thus, in the present modification, every time the image pickup device 31 moves between organs, the organ model, corresponding to the organ to which the image pickup device 31 moves, is displayed, and this makes it easy to observe the observation target region. Note that the example in FIG. 14 shows an example of detecting the movement of the distal end portion 33c to a next organ based on the image of the changeover portion. However, various methods can be adopted as the method of detecting the movement of the distal end portion 33c between organs. For example, for detecting the movement from the esophagus to the stomach, a lumen size may be obtained, and when the lumen size is changed to a size of prescribed times, the movement from the esophagus to the stomach may be detected.

Although a display direction of the organ model display is not shown in the example in FIG. 15, the organ model display, in which the top and the bottom of the organ model to be displayed are matched with the top and the bottom of the examination screen, respectively, is displayed in the same way as in the first embodiment.

### (Modification)

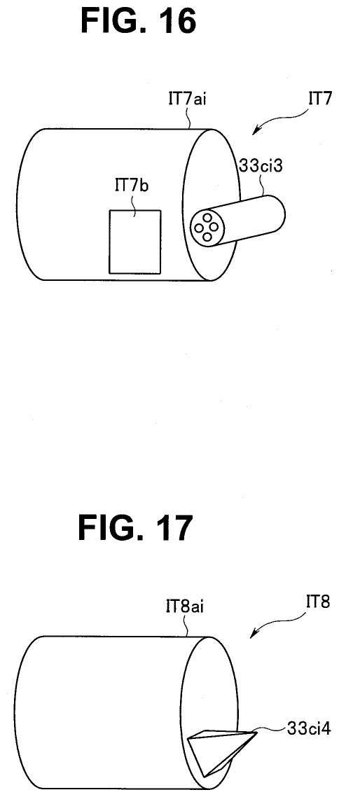

FIGS. 16 and 17 are explanatory views showing a modification. FIG. 16 shows the organ model display in the above embodiment and each of the modifications, onto which a display indicating a photographing region by the image pickup device 31 is added. FIG. 17 shows the organ model display in the above embodiment and each of the modifications, onto which a display indicating a current position and posture of the distal end of the endoscope is added.

The display content control unit 27 creates display data for organ model displays that are shown in FIGS. 16 and 17. In FIG. 16, an organ model display IT7 includes an organ model IT7ai of the lumen, an image 33ci3 of the distal end portion 33c, and an image IT7bi of the photographing region. The organ model display in FIG. 16 makes it easy for the operator to recognize the current photographing region.

In FIG. 17, an organ model display IT8 includes an image of an organ model IT8ai of the lumen, an image 33ci4 of the distal end portion 33C. In the image 33ci4, a bottom surface of a quadrangular pyramid represents a plane parallel to the image pickup surface of the image pickup device 31. For example, when the insertion portion 33 is inserted into the lumen, a central axis of the distal end portion 33c is arranged in a direction extending from an apex of the quadrangular pyramid to a center of the bottom surface, and this means that the image pickup direction of the image pickup device 31 extends from the apex of the quadrangular pyramid to the center of the bottom surface. The organ model display in FIG. 17 makes it easy for the operator to recognize the current insertion direction of the distal end of the endoscope 30 and the photographing direction. Although the distal end portion 33c is shown by the quadrangular pyramid in FIG. 17, the distal end portion 33c may be shown by any shape. For example, an image of a shape corresponding to an actual shape of the distal end portion 33c may be displayed.

### (Modification)

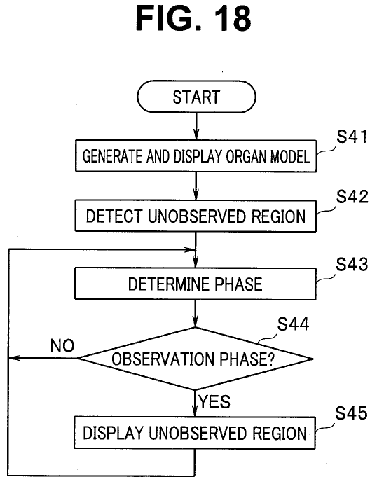

FIG. 18 is a flowchart showing a modification. A hardware configuration of the present modification is similar to the hardware configuration of the first embodiment. The modification is to control on/off of the display of an unobserved region.

In step S41 of FIG. 18, the model generation unit 25 generates an organ model. The display content control unit 27 creates display data for the organ model display. The organ model is displayed on the display screen 60a of the monitor 60. The unobserved region determination unit 26 detects an unobserved region in step S42.

In step S43, the display content control unit 27 determines whether a current phase is an observation phase for observing the organ and searching for candidates of a lesion portion, a diagnosis phase for diagnosing the lesion portion, or a treatment phase for treating the lesion portion. For example, the display content control unit 27 may determine the diagnosis phase when a distance between the image pickup device 31 and the photographing target is equal to or less than a prescribed threshold. The display content control unit 27 may also determine the diagnosis phase or the treatment phase when the moving speed of the image pickup device 31 is equal to or less than a prescribed threshold speed.

When the display content control unit 27 determines the observation phase as a result of the phase determination (YES is determined in S44), the display content control unit 27 displays an image of the unobserved region superimposed on the organ model image in step S45. When the display content control unit 27 determines the diagnosis phase or the treatment phase (No is determined in S44), the display content control unit 27 does not display the image of the unobserved region.

This can prevent the display of the unobserved region from hindering recognition of the lesion portion when diagnosis or treatment is performed.

### Second Embodiment

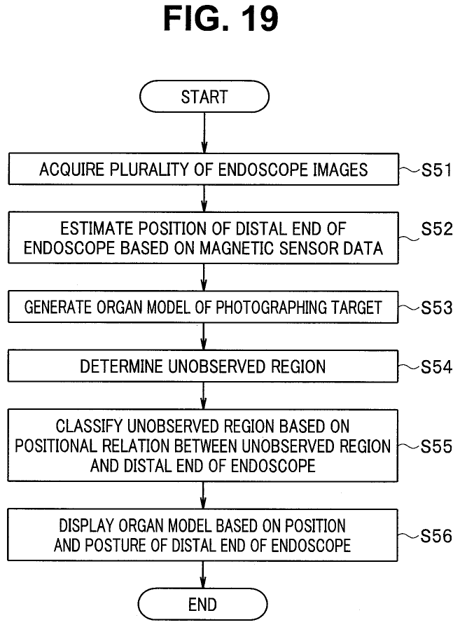

FIG. 19 is a flowchart showing a second embodiment of the present invention. A hardware configuration of the present embodiment is similar to the hardware configuration of the first embodiment in FIGS. 1 to 3. The present embodiment is configured to classify unobserved regions based on prescribed rules, and control the display of the unobserved regions according to classification results. While the display direction of the three-dimensional organ model is controlled so as to make it easier to grasp the position of the unobserved region in the first embodiment, the present embodiment is configured to make it easy to grasp the position of unobserved regions for each type on the organ model or the examination screen.

In the present embodiment, the unobserved regions are divided into four classification items to optimize the display. The four classification items include (1) an occlusion region, (2) a short observation time region, (3) a photographed region, and (4) an examination screen outside region. This allows the operator to grasp a cause of the unobserved regions or the like, and may be able to help determination of the position to be observed next, for example.

(1) The occlusion region is an unobserved region due to shielding objects. Examples of the occlusion region may include regions behind folds or regions occluded by residues or bubbles.

(2) The short observation time region refers to a region that is not observable due to fast moving speed of the distal end of a scope.

(3) The photographed region is a region that has been photographed from among the unobserved regions.

(4) The examination screen outside region is an unobserved region outside the examination screen that exists outside a current image pickup range.

The CPU 21 in the processor 20 classifies an unobserved region into at least one or more of the regions (1) to (4). The CPU 21 acquires information on an unobserved region from the unobserved region determination unit 26, and acquires the position and posture information on the image pickup device 31 from the position and posture estimation unit 24 so as to classify the unobserved region based on the position and the posture of the image pickup device 31. The CPU 21 provides the classification result of each region to the display content control unit 27. The display content control unit 27 creates the display data in a display form that is set for each of the unobserved regions (1) to (4).

### (Occlusion Region)

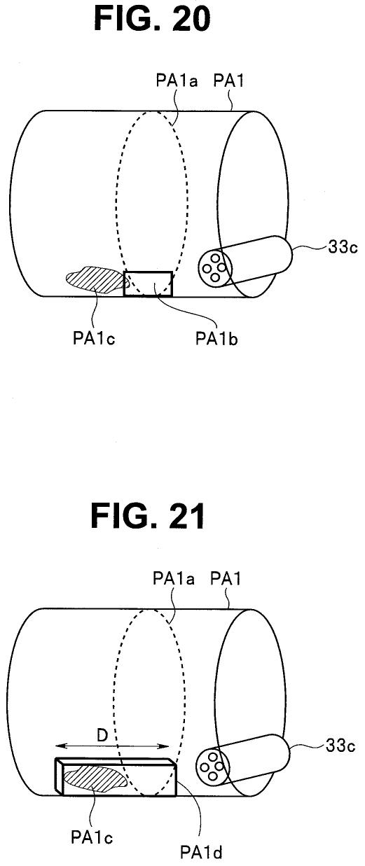

FIGS. 20 and 21 are explanatory view for describing a detection method of the occlusion region.

The CPU 21 detects the regions (hereafter referred to as occlusion regions) that are likely to be occluded due to the presence of occlusion elements that shield the visual field, such as folds. For example, the CPU 21 detects folds inside the lumen to be examined, and elements such as residues, bubbles, and bleeding present inside the lumen as occlusion elements. For example, the CPU 21 may use AI to determine the occlusion elements. For example, an inference model is generated in advance by acquiring a plurality of examination images including occlusion elements, and performing deep learning using the examination images as teacher data. The CPU 21 may use the inference model to determine the occlusion elements and the occlusion regions in the examination images.

FIG. 20 shows an example in which a fold PA1a is present in a lumen PA1, and an unobserved region PA1c is caused by the fold PA1a that is an occlusion element PA1b. The CPU 21 sets a search region that is in a prescribed distance D in a direction opposite to a direction extending from the occlusion element to the distal end of the distal end portion 33c. FIG. 21 shows a framed search region PA1d. The CPU 21 sets the unobserved region PA1c present in the search region PA1d as an occlusion region. Note that the CPU 21 may change the setting of the distance D for each occlusion element. The CPU 21 provides information about the occlusion region to the display content control unit 27. The display content control unit 27 displays a display indicating the occlusion region in the examination screen.

There is also a method of detecting the occlusion region using a region already determined as an unobserved region. With reference to FIG. 20 for description, when the occlusion element PA1b is detected in a region between the unobserved region PA1c and the endoscopic position, the unobserved region PA1c is classified as an occlusion region. Searching for the occlusion elements based on the unobserved region has an advantage that the amount of computation can be reduced more.

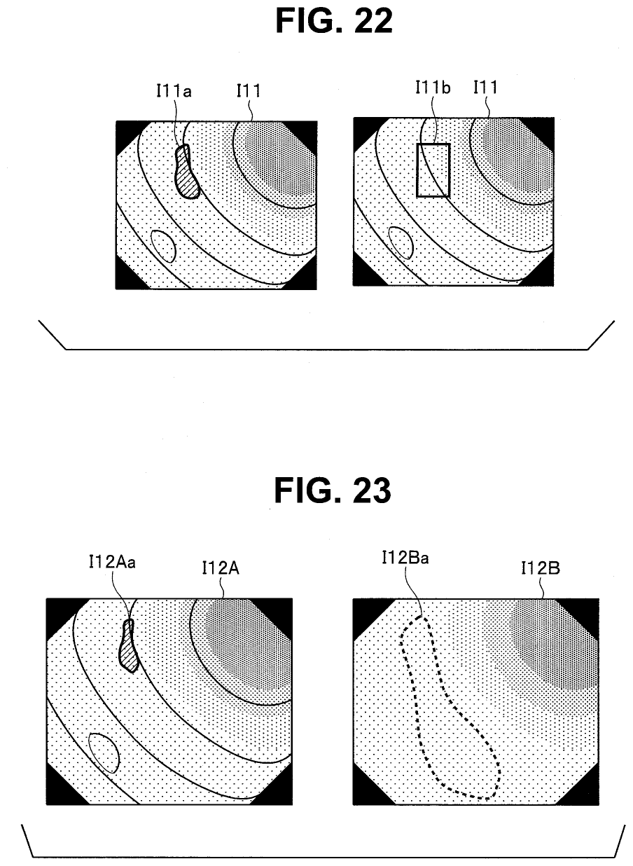

FIG. 22 is an explanatory view showing an example of the display method of the occlusion region by the display content control unit 27.

A left side in FIG. 22 shows an example in which an occlusion region i11a present in an examination screen I11 is displayed by hatching. For example, the occlusion region I11a may be displayed by a contour frame line or a rectangular frame line, or may be painted and displayed. A right side in FIG. 22 shows an example of an occlusion region I11b displayed by a rectangular frame line. The display content control unit 27 may also display the occlusion region in a display color corresponding to each occlusion element.

### (Short Observation Time Region)

The CPU 21 calculates the moving speed of the image pickup device 31 from the frame rate of the examination image and changes in position of the distal end portion 33c, for example. The CPU 21 acquires information on the position of the image pickup device 31 from the position and posture estimation unit 24, and acquires information on a position of an unobserved region from the unobserved region determination unit 26 to obtain the moving speed of the image pickup device 31 that passes the unobserved region.

When the moving speed of the image pickup device 31 that passes the unobserved region is equal to or more than a prescribed threshold, the CPU 21 classifies the unobserved region as a short observation time region. The CPU 21 provides information about the short observation time region to the display content control unit 27. The display content control unit 27 displays a display indicating the short observation time region in the examination screen.

The display content control unit 27 displays the short observation time region in a display format in conformity with the display methods of other classifications that are classified. In this case, the display content control unit 27 displays the short observation time region by changing a display color or a line type (solid line/dotted line) so as to be recognized as the short observation time region.

### (Photographed Region)

The CPU 21 acquires information about an unobserved region from the unobserved region determination unit 26. The unobserved region determination unit 26 sequentially determines the unobserved region. Based on the information from the unobserved region determination unit 26, the CPU 21 can grasp that the unobserved region has changed to the photographed region. The CPU 21 provides information about the photographed region to the display content control unit 27. The display content control unit 27 displays a display indicating the photographed region. The CPU 21 may also notify a user at predetermined timing that the unobserved region has changed to the photographed region.

Note that the CPU 21 may be configured to inform the operator of the position of the unobserved region. When the operator moves the insertion portion 33 and photographs an unobserved region with the image pickup device 31, the unobserved region is classified to the photographed region.

FIG. 23 is an explanatory view showing an example of a display method of the photographed region by the display content control unit 27.

A left side in FIG. 23 shows an example in which an unobserved region I12Aa, such as an occlusion region present in an examination screen I12A, is displayed by hatching. An examination screen I12B on a right side in FIG. 23 indicates by a dashed frame line that the unobserved region I12Aa on the left side in FIG. 23 has been photographed and become a photographed region I12Ba. Note that the display content control unit 27 only needs to indicate that the unobserved region has been photographed and become the photographed region I12Ba by a display method different from the display method of the unobserved region I12A. Therefore, the display method is not limited to the method in FIG. 23 and various display methods can be adopted.

### (Examination Screen Outside Region)

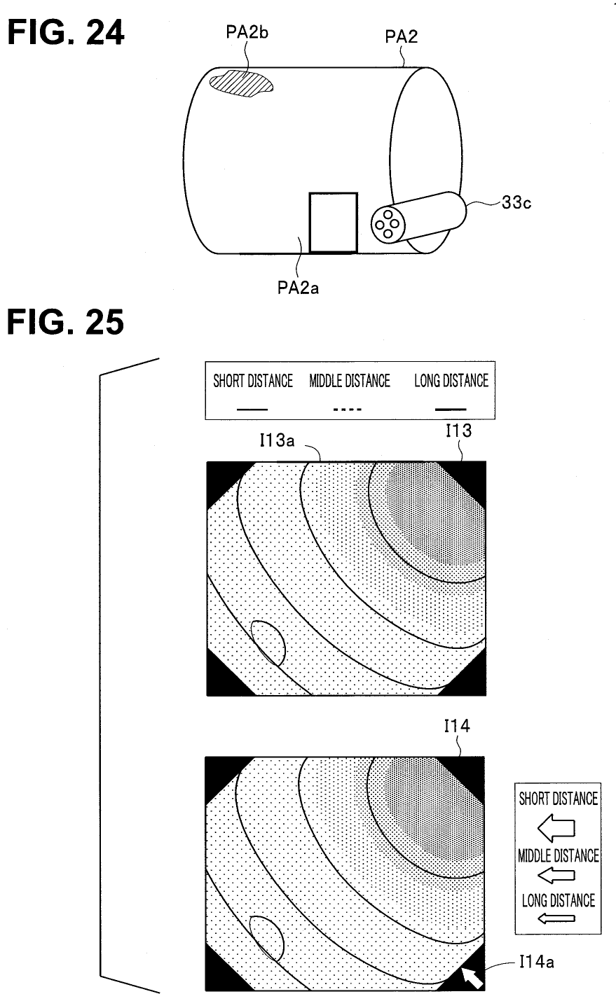

FIG. 24 is an explanatory view for describing the examination screen outside region.

FIG. 24 shows a state where an image of the inside of a lumen PA2 is picked up by the image pickup device 31 in the distal end portion 33c. A rectangular frame in the lumen PA2 indicates an image pickup range PA2a of the image pickup device 31. In other words, in the example of FIG. 24, the unobserved region shown by hatching is outside the image pickup range PA2a, that is, an examination screen outside region PA2b that is present outside the examination screen obtained by the image pickup device 31.

When the CPU 21 is provided with information about the photographing region and the unobserved region from the model generation unit 25 and the unobserved region determination unit 26, the CPU 21 classifies the unobserved region outside the photographing region as the examination screen outside region. The CPU 21 outputs the classification result of the examination screen outside region to the display content control unit 27. The display content control unit 27 displays a display indicating the examination screen outside region.

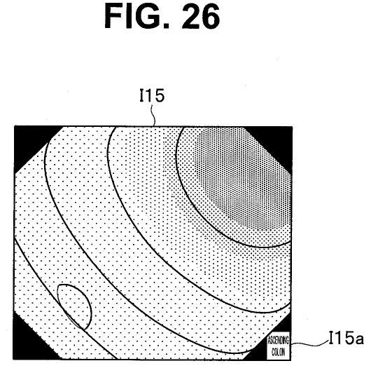

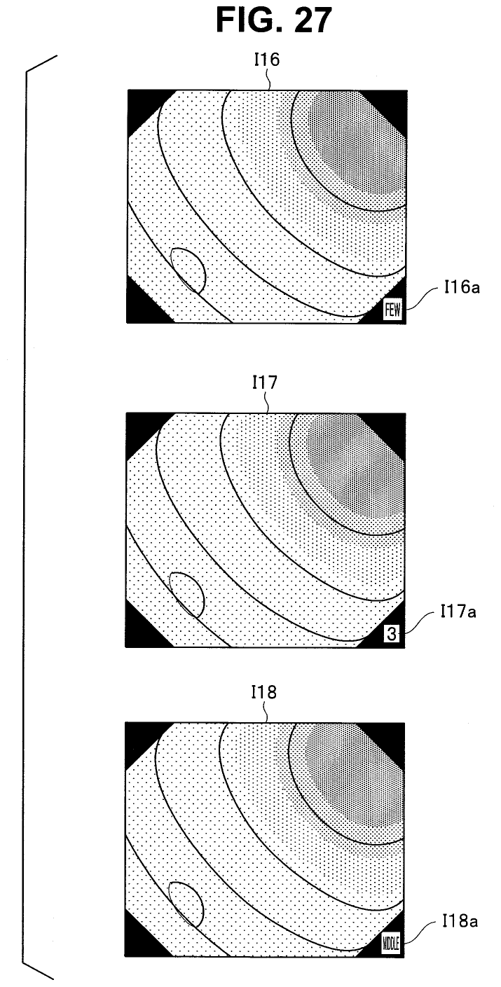

FIGS. 25 and 26 are explanatory views showing an example of the display method of the examination screen outside region.

An upper stage of FIG. 25 shows an example of displaying a direction and a distance of an examination screen outside region present outside an examination screen I13 by a line segment I13a. A display position and a type of the line segment I13a indicate the direction and the distance of the examination screen outside region. In other words, the direction of the examination screen outside region is indicated by which side, of four sides of the examination screen I13, the line segment I13a is arranged on. A thin line, a dashed line, and a thick line of the line segment I13a indicate whether the examination screen outside region is in a short distance, a middle distance, or a long distance from the photographing range. The example in the upper stage of FIG. 25 indicates that the examination screen outside region is present in a top direction of the photographing range (the examination screen) and in the long distance. Note that the CPU 21 can change threshold values of the short distance, the middle distance, and the long distance as appropriate. Note that the distance and the direction to the examination screen outside region can be expressed by changing parameters such as a color, brightness, a thickness, a length, and a type of the line segment.

A lower stage in FIG. 25 shows an example of displaying a direction and a distance of an examination screen outside region present outside an examination screen I14 by an arrow I14a. A direction and a thickness of the arrow I14a indicate the direction and the distance of the examination screen outside region. In other words, the direction of the arrow I14a indicates the direction of the examination screen outside region. The thickness of the arrow I14a indicates whether the examination screen outside region is in a short distance, a middle distance, or a long distance from the photographing range. The example in the lower stage of FIG. 25 shows that the thicker the arrow I14a is, the shorter the distance is. The example in the lower stage of FIG. 25 indicates that the examination screen outside region is present in an obliquely top direction of the photographing range (the examination screen) and in the middle distance. Note that the CPU 21 can change the threshold values of the short distance, the middle distance, and the long distance as appropriate. Note that the distance and the direction to the examination screen outside region can be expressed by changing parameters such as a color, brightness, a thickness, a length, and a type of the arrow. Although FIG. 25 shows an example of indicating the direction of the examination screen outside region based on the photographing range, a route from the photographing range to the examination screen outside region may be displayed.

FIG. 26 shows an example showing a name of an organ where the examination screen outside region is located. In FIG. 26, a position of the examination screen outside region present outside an examination screen I15 is expressed by the name of the organ where the examination screen outside region is present. In the example of FIG. 26, an organ name display I15a indicates the presence of the examination screen outside region in a part of an ascending colon.

The display content control unit 27 may also display various types of information about the examination screen outside region on the examination screen. The CPU 21 acquires the various types of information about the examination screen outside region by acquiring information from the unobserved region determination unit 26, and outputs the acquired information to the display content control unit 27. The display content control unit 27 displays on the examination screen a display based on the information from the CPU 21.

FIG. 27 is an explanatory view showing a display example of the various types of information about the examination screen outside region.

An upper stage in FIG. 27 shows an example of displaying the number of the examination screen outside regions that are present outside an examination screen I16, by category. For example, the display content control unit 27 displays a category display I16a of “many” when there are five or more examination screen outside regions, and displays a category display I16a of “few” when there are less than five examination screen outside regions. The example in the upper stage of FIG. 27 shows that there are less than five examination screen outside regions.

A middle stage in FIG. 27 shows an example of displaying an absolute number display I17a of the number of the examination screen outside regions that are present outside an examination screen I17. The example in the middle stage in FIG. 27 shows that there are three examination screen outside regions.

A lower stage in FIG. 27 shows an example of displaying a size of the examination screen outside region present outside an examination screen I18, by category. For example, the display content control unit 27 may divide the size of the examination screen outside region into three stages and display a category display I18a of “small”, “medium, and “large” from a small size to a large size. The example in the lower stage of FIG. 27 shows that the size of the examination screen outside region is a middle size.

Next, the operation of the thus-configured embodiment is described by referring to FIG. 19.

After power is applied to the endoscope apparatus 1, the insertion portion 33 is inserted into an examination target, and examination is started. The image pickup device 31 is driven by the image generation circuit 40 to pick up images of an inside of a patient and acquire a plurality of endoscope images (step S51). Image signals from the image pickup device 31 are supplied to the image generation circuit 40 for prescribed image processing. The image generation circuit 40 generates examination images (endoscope images) based on the image signals and outputs the examination images to the monitor 60. In this way, the examination images are displayed on the display screen 60a of the monitor 60.

The position and posture detection unit 12 estimates the position of the distal end of the endoscope using magnetic sensor data from the magnetic field generation apparatus 50 (step S52). The position and the posture of the distal end of the endoscope estimated by the position and posture detection unit 12 are supplied to the processor 20. The examination image from the image generation circuit 40 is also supplied to the image processing apparatus 10. The image acquisition unit 11 supplies the received examination image to the processor 20. The examination image is supplied to the model generation unit 25 by the input/output unit 23 of the processor 20. The model generation unit 25 generates an organ model in step S53 (step S53).

The unobserved region determination unit 26 determines an unobserved region surrounded with the organ model generated by the model generation unit 25 (step S54), and outputs the determination result to the CPU 21 and the display content control unit 27. The CPU 21 classifies the unobserved region to an occlusion region, an short observation time region, a photographed region, and an examination screen outside region, based on the positional relation between the unobserved region and the distal end of the endoscope, and outputs the classification result to the display content control unit 27. The display content control unit 27 displays the examination screen and the organ model on the display screen 60a of the monitor 60 according to the classification result (step S56).

Thus, in the present embodiment, the unobserved regions are classified into four classification items including the occlusion region, the short observation time region, the photographed region, and the examination screen outside region, and displayed. This allows the operator to grasp a cause or the like of the unobserved region, and can help determination of a position to be observed next, for example.

### Third Embodiment

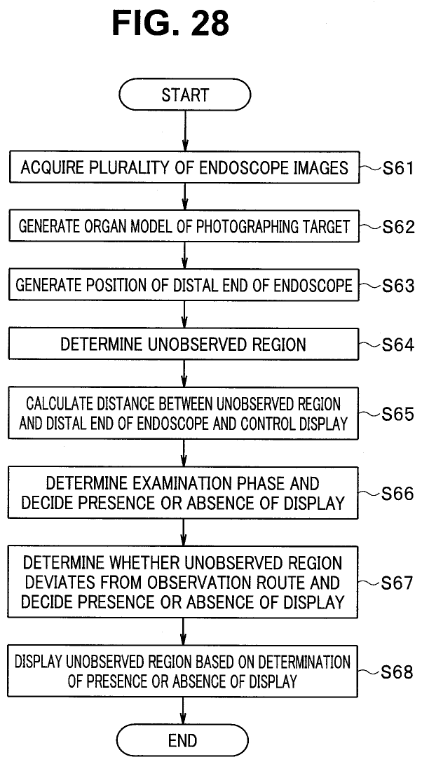

FIG. 28 is a flowchart showing a third embodiment in the present invention. A hardware configuration of the present embodiment is similar to the hardware configuration of the first embodiment in FIGS. 1 to 3. The present embodiment is configured to control the display of the unobserved region based on the relation among a distance from the distal end of the endoscope, an examination phase, and an observation route.

The CPU 21 calculates a distance (Euclidean distance) between an unobserved region and the distal end of the endoscope for display control of the unobserved region. The CPU 21 acquires information on a position of the distal end of the endoscope from the position and posture estimation unit 24, and acquires information on a position of the unobserved region from the unobserved region determination unit 26, so as to obtain the distance between the unobserved region and the distal end of the endoscope.

The CPU 21 also determines diagnosis and treatment phases for display control of the unobserved region, and determines places where insertion and removal of the insertion portion 33 is difficult. For example, the CPU 21 generates an inference model by performing deep learning with examination images in the places having high operation difficulty, such as insertion and removal, as teacher data, and uses the inference model so that the places having high operation difficulty can be determined. The CPU 21 may also determine the diagnosis or treatment phase, which requires an intensive work of the operator, by such a method as determining treatment instruments by using an operating signal from the endoscope 30 or using AI. The CPU 21 also acquires information on the observation route for display control of the unobserved region. For example, the CPU 21 can determine which position on the observation route is currently under observation by storing the information on the observation route in the storage unit 22 and using outputs of the position and posture estimation unit 24, the model generation unit 25, and the unobserved region determination unit 26. The CPU 21 may also output, to the user, information on an operation method to reach an unobserved region. For example, the CPU 21 may output information, such as raising the distal end of the endoscope, pulling the endoscope back, or pushing the endoscope in, for example.

The CPU 21 outputs acquired various types of information to the display content control unit 27. The display content control unit 27 controls the display of the unobserved region based on the various types of information from the CPU 21.

### (Distance Display Control)

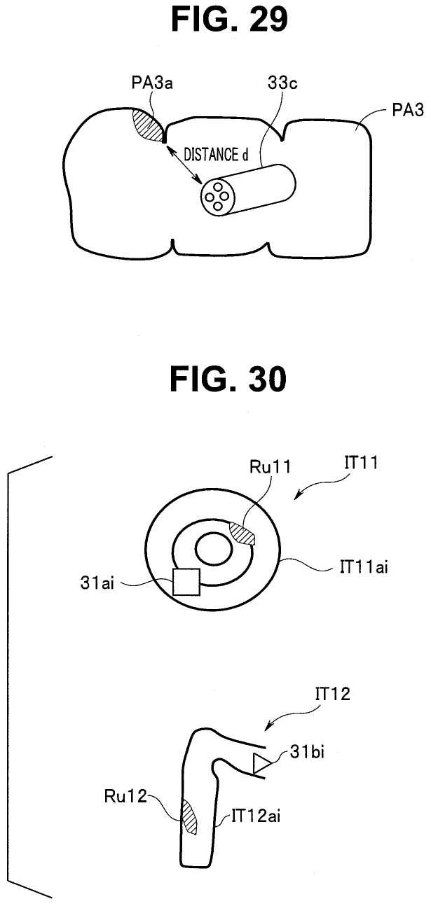

FIG. 29 is an explanatory view showing a state where an image of an inside of a lumen PA3 is picked up by the image pickup device 31 at the distal end portion 33c. In the lumen PA3, there is an unobserved region PA3a indicated by hatching. The CPU 21 calculates, as a distance d between the image pickup device 31 and the unobserved region PA3a, an Euclidean distance between coordinates of the unobserved region PA3a calculated during model generation and coordinates of the distal end of the endoscope. The CPU 21 outputs the acquired information on the distance d to the display content control unit 27. The CPU 21 also generates a threshold θ to decide display on/off and outputs the threshold θ to the display content control unit 27.

In a process of organ model generation, a large number of unobserved regions emerge in the vicinity of the image pickup device 31, and when the regions in the vicinity of the image pickup device 31 are displayed on the examination screen as unobserved regions, visibility of an observation site may deteriorate, and this may cause difficulty in observation. Therefore, in the present embodiment, unobserved regions present at a distance closer than the threshold θ are controlled so as not to be displayed.

The display content control unit 27 is provided with the information about unobserved regions from the unobserved region determination unit 26, and is also provided with the distance d and the threshold θ for each of the unobserved regions from the CPU 21. The display content control unit 27 displays the unobserved region on the examination screen when the distance d to the pertinent unobserved region exceeds the threshold θ.

The CPU 21 also reduces the threshold value θ before the image pickup device 31 passes through a site having high operation difficulty. For example, in examination of a colon, the threshold θ is reduced in front of sites having difficulty in insertion or removal of the insertion portion 33, such as a sigmoid colon and splenic flexure portions. As a result, the unobserved regions at a relatively short distance from the image pickup device 31 are also displayed on the examination screen. The operator performs bending operation of the insertion portion 33 so that the unobserved regions are eliminated. As a result, the unobserved regions are less overlooked in such sites, and repeated insertion and removal of the insertion portion 33 can be prevented.

The CPU 21 also reduces the threshold θ immediately after diagnosis or treatment. During operation such as diagnosis and treatment, the operator often concentrates on a work in a fixed place for a fixed period of time. In such a case, it is possible for the operator to forget to observe the unobserved region that the operator remembered to observe before the work on such a place. Accordingly, immediately after such a treatment work, the unobserved regions at a relatively short distance from the image pickup device 31 are also displayed. For example, the CPU 21 recognizes the phases and reduces the threshold θ by such methods as switching between normal-light observation and NBI (narrow band) observation, detection of zoom-in and zoom-out operations, and detection of treatment instruments using AI.

### (Viewpoint Control)

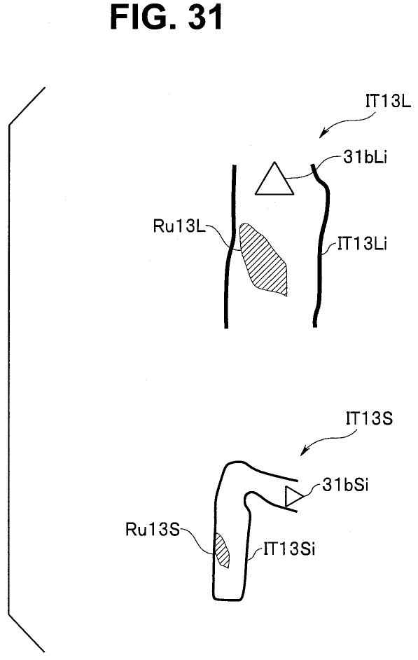

FIG. 30 is an explanatory view for describing viewpoint control in accordance with the distance d. The display content control unit 27 may control the display viewpoint of the organ model according to the distance d.

An upper stage in FIG. 30 shows an organ model display IT11 when the distance d is relatively small, and a lower stage in FIG. 30 shows an organ model display IT12 when the distance d is relatively large. The organ model display IT11 shown in the upper stage in FIG. 30 includes an image IT11ai of the organ model of a lumen, an image 33ai of the image pickup device 31, and an image Ru11 of an unobserved region, and the organ model display IT11 is displayed from the viewpoint of a travel direction of the image pickup device 31.

The organ model display IT12 shown in the lower stage in FIG. 30 includes an image IT12ai of the organ model of a lumen, an image 33bi of the image pickup device 31, and an image Ru12 of an unobserved region, and the organ model display IT12 is displayed from the viewpoint of bird's eye.

### (Magnification Rate Control)

FIG. 31 is an explanatory view for describing magnification rate control in accordance with the distance d. The display content control unit 27 may control the magnification rate of the organ model according to the distance d.

An organ model display IT13L in an upper stage in FIG. 31 shows a display when the distance d between the image pickup device 31 and the unobserved region is relatively small, and an organ model display IT13S shows a display when the distance d between the image pickup device 31 and the unobserved region is relatively large.

The organ model displays IT13S and IT13L are based on, for example, the organ model of an intestinal tract of the same subject. The organ model display IT13S indicates that the organ model in a relatively wide range, from the distal end of the organ model to substantially the position of the image pickup device 31, is displayed at a relatively small display magnification rate. The organ model display IT13S includes an image IT13Si of the organ model, an image 31bSi of the image pickup device 31, and an image Ru13S of an unobserved region.

The organ model display IT5L indicates that a relatively narrow range of an organ model in the vicinity of the image pickup device 31 is displayed at a relatively large display magnification rate. The organ model display IT13L includes an image IT13Li of the organ model, an image 31bLi of the image pickup device 31, and an image Ru13L of an unobserved region.

### (Emphasis)

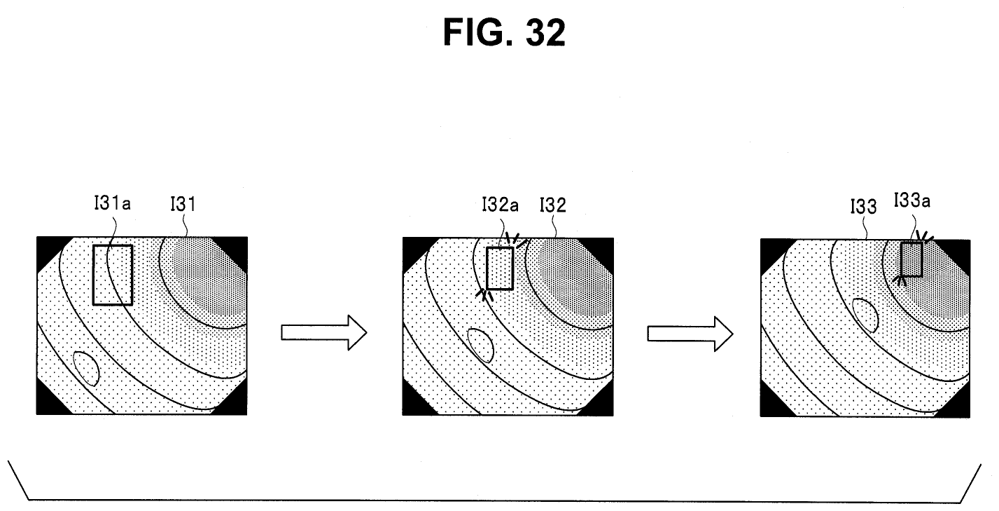

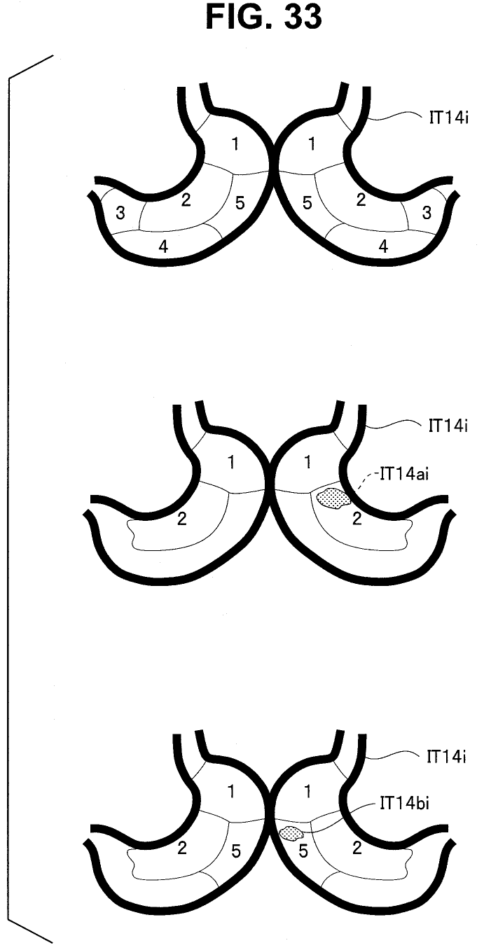

FIG. 32 is an explanatory view for describing emphasis display in accordance with a distance. The display content control unit 27 may control an emphasis degree of an unobserved region according to the distance d.

A left side in FIG. 32 shows an unobserved region present in an examination screen I31 by a square frame image I31a. The distance d between the unobserved region and the image pickup device 31 changes as the image pickup device 31 moves. A center portion of FIG. 32 shows an example of an examination screen I32 in this case, that is, an example where the distance d increases due to the movement of the image pickup device 31. When the distance d is equal to or more than a first threshold, the display content control unit 27 may display a square frame image I32a indicating an unobserved region by blinking.

It is assumed that the distance d is further increased by the movement of the image pickup device 31. A right side in FIG. 32 shows a display example of an examination screen I33 in this case. When the distance d is equal to or more than the first threshold, the display content control unit 27 may increase a blinking speed of a square frame image I33a that indicates an unobserved region, according to the distance d.

Such emphasis display of the unobserved region prevents the operator from overlooking the unobserved region. Note that in addition to the blinking control, various emphasis displays, such as changing brightness or changing a thickness of the square frame, may also be adopted.

(Deviation from Observation Route)

FIG. 33 is an explanatory view for describing display control in accordance with the observation route. The display content control unit 27 may perform display control of the organ model according to the observation route.

The CPU 21 acquires information on the observation route of an organ and outputs the acquired information to the display content control unit 27. The display content control unit 27 compares an observation route order of the region at the position of the distal end of the endoscope and an observation route order at the position of the unobserved region, and when the observation route order at the position of the unobserved region is latter, the pertinent unobserved region is not displayed.

FIG. 33 shows an organ model display IT14i of a stomach. Sections in the organ model display IT14i and the number of each section indicate the order of the observation route, and the display of the sections and the number is omitted on the screen. In a middle stage in FIG. 33, a hatched image IT14ai shows that an unobserved region is present in a section 2, and in a lower stage in FIG. 33, a hatched image IT14bi shows that an unobserved region is present in a section 5.

Thus, an unobserved region is present in the section 2 and the section 5. Observation by the image pickup device 31 is performed in the order of the section number starting from the section 1. Assume that the image pickup device 31 is now located in the section 2 and is observing the region of the section 2. In this case, as shown in the middle stage of FIG. 33, the display content control unit 27 displays the image IT14ai indicating the unobserved region in the section 2, and does not display the unobserved region in the section 5 that is latter in the observation route order than the section that is currently observed.

Assume that as the observation proceeds, the image pickup device 31 reaches the section 5 and is in a state of observing the region in the section 5. In this case, as shown in the lower stage in FIG. 33, the display content control unit 27 displays the image IT14bi indicating the unobserved region in the section 5.

In this way, since the display of the unobserved regions is controlled according to the observation route, smooth observation is easily performed.

In the example shown in FIG. 33, the unobserved region is not displayed for the section that is latter in the observation route order than the section that is currently observed. However, a method such as displaying the unobserved region with a low brightness may be adopted.

Next, the operation of the thus-configured embodiment is described by referring to FIG. 28.

After power is applied to the endoscope apparatus 1, the insertion portion 33 is inserted into an examination target, and examination is started. The image pickup device 31 is driven by the image generation circuit 40 to pick up images of an inside of a patient and acquire a plurality of endoscope images (step S61). Image signals from the image pickup device 31 are supplied to the image generation circuit 40 for prescribed image processing. The image generation circuit 40 generates examination images (endoscope images) based on the image pickup signals and outputs the examination images to the monitor 60. In this way, the examination images are displayed on the display screen 60a of the monitor 60.

The examination images from the image generation circuit 40 are also supplied to the image processing apparatus 10. The image acquisition unit 11 supplies the received examination images to the processor 20. The examination images are supplied to the position and posture estimation unit 24 and the model generation unit 25 by the input/output unit 23 of the processor 20. The model generation unit 25 generates an organ model (step S62), and the position and posture estimation unit 24 obtains a position of the distal end of the endoscope (step S63).

The unobserved region determination unit 26 determines an unobserved region surrounded with the organ model generated by the model generation unit 25 (step S64), and outputs the determination result to the CPU 21 and the display content control unit 27. The CPU 21 calculates the distance d between the unobserved region and the distal end of the endoscope based on the positional relation between the unobserved region and the distal end of the endoscope, and obtains the threshold θ. The CPU 21 outputs the distance d and the threshold θ to the display content control unit 27 to control the display (step S65).

The CPU 21 determines an examination phase and provides the determination result to the display content control unit 27 to control the display (step S66). In addition, the CPU 21 determines whether each of the unobserved regions is an unobserved region that deviates from the observation route, and provides the determination result to the display content control unit 27 to control the display (step S67). The display content control unit 27 controls the display based on the output of the CPU 21 (step S68). For steps S65 to S68, at least one of the processes may be executed, and the execution order is not particularly limited.

Thus, in the present embodiment, the display of the unobserved regions is controlled based on the distance to the distal end of the endoscope, the examination phase, and the observation route, which provides an effect of making it easier for the operator to perform observation based on the examination screen and the organ model.

The present invention is not limited to each of the embodiments in its entirety, but may be embodied by modifying the component members without departing from the meaning of the invention during an implementation stage. Moreover, various modes of the present invention may be formed by appropriately combining a plurality of the component members disclosed in each of the embodiments. For example, some component members out of all the component members shown in the embodiments may be deleted. Furthermore, component members in different embodiments may properly be combined.

What is claimed is:

1. An image processing apparatus, comprising a processor, wherein
the processor is configured to
acquire image information from an endoscope during observation of an inside of a subject,
generate an organ model from the acquired image information,
identify an unobserved region that is not observed by the endoscope in the organ model,
estimate a top and a bottom and an azimuth of an image pickup visual field of the endoscope with respect to the organ model,
set a display direction of the organ model based on the top and the bottom and the azimuth of the image pickup visual field, and
output the organ model to a monitor, the organ model being associated with the unobserved region identified.

2. The image processing apparatus according to claim 1, wherein the processor matches a top and bottom direction of the organ model with a top and bottom direction of an observation image of the endoscope.

3. The image processing apparatus according to claim 1, wherein
the processor performs viewpoint direction control that rotates the organ model that is currently displayed in matching with a viewpoint of the endoscope.

4. The image processing apparatus according to claim 1, wherein the processor displays a photographing region on the organ model.

5. The image processing apparatus according to claim 1, wherein the processor displays information on positional relation between the unobserved region and the endoscope.

6. The image processing apparatus according to claim 5, wherein the information on positional relation is information indicating a route from a distal end position of the endoscope to the unobserved region.

7. The image processing apparatus according to claim 5, wherein the information on positional relation is information indicating a distance from a distal end position of the endoscope to the unobserved region.

8. The image processing apparatus according to claim 1, wherein the processor outputs, to a notification unit, endoscope operation information for a user to move the endoscope from a current position to the unobserved region.

9. The image processing apparatus according to claim 1, wherein the processor displays a name of an organ where the unobserved region is located.

10. The image processing apparatus according to claim 1, wherein the processor displays the number of or area of the unobserved region or unobserved regions.

11. An endoscope apparatus, comprising:
an endoscope;
an image processing apparatus including a processor; and
a monitor, wherein
the processor is configured to
acquire image information from the endoscope during observation of an inside of a subject,
generate an organ model from the acquired image information,
identify an unobserved region that is not observed by the endoscope in the organ model,
estimate a position and a posture of the endoscope with respect to the organ model,
set a display direction of the organ model based on the position and the posture of the endoscope, and
output the organ model to the monitor, the organ model being associated with the unobserved region identified.

12. An image processing method, comprising:
acquiring image information from an endoscope during observation of an inside of a subject;
generating an organ model from the image information acquired;
identifying an unobserved region that is not observed by the endoscope in the organ model;
estimating a position and a posture of the endoscope with respect to the organ model;
setting a display direction of the organ model based on the position and the posture of the endoscope; and
outputting the organ model to a monitor, the organ model being associated with the unobserved region identified.
